(19) United States
(12) Patent Application Publication
(10) Pub. No.: US 2023/0335241 A1
(43) Pub. Date: Oct. 19, 2023

(54) MEDICAL INFORMATION PROCESSING APPARATUS AND METHOD

(71) Applicant: Canon Medical Systems Corporation, Otawara-shi (JP)

(72) Inventors: Koichi TERAI, Shioya (JP); Yasunari KUTSUNA, Nasushiobara (JP); Kyohei NORO, Nasushiobara (JP); Jyunichi YOSHIDA, Nasushiobara (JP); Ryoichi ONO, Utsunomiya (JP); Guang Yi ONG, Nasushiobara (JP)

(73) Assignee: Canon Medical Systems Corporation, Otawara-shi (JP)

(21) Appl. No.: 18/299,787

(22) Filed: Apr. 13, 2023

(30) Foreign Application Priority Data
Apr. 14, 2022 (JP) 2022-066926
Apr. 12, 2023 (JP) 2023-064957

(51) Int. Cl. G16H 10/60; G16H 50/30

(52) U.S. Cl. CPC G16H 10/60; G16H 50/30; A61B 5/0002

(57) ABSTRACT

The medical information processing apparatus includes a processor. Through a channel opened on a chat application and used for discussing a target medical care, the processor obtains a record of messages that have been entered by the channel members. The processor obtains role information, which represents the role of a target member among the channel members. The processor also obtains progress information, which represents the current progress of the target care. The processor identifies one or more candidates for a message to be entered by the target member based on the message record, role information and/or progress information. The processor displays the one or more candidates on an input screen relating to the channel of the target member.

### CROSS-REFERENCE TO RELATED APPLICATIONS

This application is based upon and claims the benefit of priority from the Japanese Patent Application No. 2022-066926, filed Apr. 14, 2022, and No. 2023-64957, filed Apr. 12, 2023, the entire contents of all of which are incorporated herein by reference.

### FIELD

Embodiments described herein relate generally to a medical information processing apparatus and method.

### BACKGROUND

For diseases such as cerebral embolism and pulmonary embolism, international guidelines recommend team medical care, where a care team is organized from different divisions so as to make the most appropriate determinations on the medical care and provide the most appropriate treatment. To assist such team medicine, applications have been offered so that the team members can share the conditions of the diagnosis and treatment and make suitable decisions. Some applications have a function of many-to-many message exchange (chatting). Depending on the conditions of the diagnosis/treatment and also on the users' predispositions, they may feel annoyed to input character strings for chatting. For diseases such as cerebral embolism and pulmonary embolism, in particular, with which every minute and second counts, a cumbersome task of inputting information may be one factor that hinders the use of the chat.

### BRIEF DESCRIPTION OF THE DRAWING

FIG. 1 is a diagram showing an exemplary network configuration of a medical information system according to the first embodiment.

FIG. 2 is a diagram showing an exemplary configuration of a medical information processing apparatus according to the first embodiment.

FIG. 3 is a diagram showing exemplary inputs and outputs of a first model.

FIG. 4 is a diagram showing an exemplary processing procedure performed by a medical information processing apparatus according to the first embodiment on a chat application.

FIG. 5 is a diagram showing an exemplary candidate message identifying process by use of a second model and a third model.

FIG. 6 is a diagram showing a transition of an exemplary input screen on a chat application in Example 1.

FIG. 7 is a diagram showing a transition of an exemplary input screen on a chat application in Example 2.

FIG. 8 is a diagram showing a transition of an exemplary input screen on a chat application in Example 3.

FIG. 9 is a diagram showing a transition of an exemplary input screen on a chat application in Example 4.

FIG. 10 is a diagram showing a transition of an exemplary input screen on a chat application in Example 5.

FIG. 11 is a diagram showing an exemplary configuration of a medical information processing apparatus according to the second embodiment.

FIG. 12 is a diagram showing an exemplary processing procedure of a chat application on the medical information processing apparatus according to the second embodiment.

FIG. 13 is a diagram showing a medical service information acquiring process through push notification.

FIG. 14 is a diagram showing a medical service information acquiring process through automatic polling.

FIG. 15 is a diagram showing a medical service information acquiring process in accordance with stepwise processing.

FIG. 16 is a diagram showing an exemplary display of the progress of medical service information obtainment.

### DETAILED DESCRIPTION

In general, according to one embodiment, a medical information processing apparatus includes a processing circuitry. Through a channel for discussing a target diagnosis and treatment opened on a chat application, the processing circuitry obtains a message record, which is a record of messages that have been input by the channel members. The processing circuitry obtains role information, which represents the role of a target member among the channel members. The processing circuitry also obtains progress information, which represents the current progress of a target care. The processing circuitry identifies one or more candidates for a message to be input by the target member based on the message record, role information, and/or progress information. The processing circuitry displays the one or more candidates on an input screen relating to the channel of the target member.

### First Embodiment

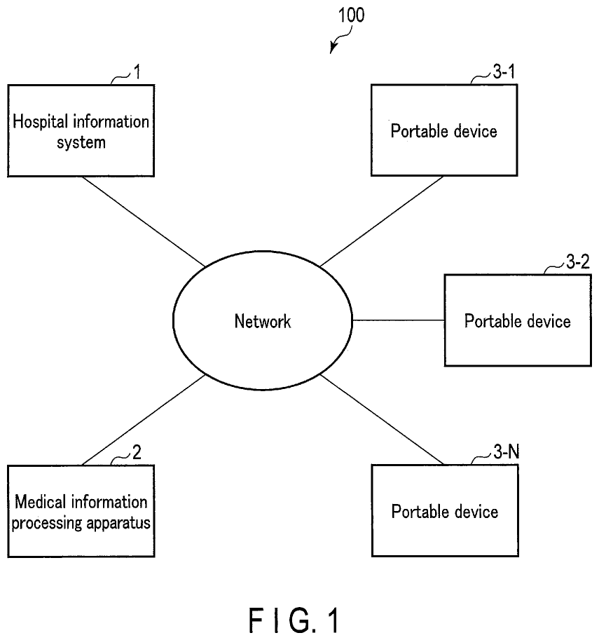

FIG. 1 shows an exemplary network configuration of a medical information system 100 according to the first embodiment. The medical information system 100 is a computer network system for sharing information of a target care among the members who constitute the care team. A target care denotes a medical care exercised by the care team. The medical care according to the present embodiment collectively refers to a medical consultation, diagnosis, and treatment. As illustrated in FIG. 1, the medical information system 100 includes a hospital information system 1, a medical information processing apparatus 2, and N portable devices 3-n (n≤1≤N, N≥1, where n is a suffix representing individual portable devices). The hospital information system 1, medical information processing apparatus 2, and N portable devices 3-n are mutually connected via a network in an information communicable manner in accordance with standards such as Digital Imaging and Communications in Medicine (DICOM). Hereinafter, if the portable devices 3-1, 3-2, . . . , and 3-N do not need to be particularly distinguished from one another, they will be referred to as portable devices 3.

The hospital information system 1 is a computer network system established in medical facilities such as a hospital. The hospital information system 1 manages various types of information relating to medical care practiced in the medical facility (hereinafter referred to as “medical service information”). The hospital information system 1 is constituted by various systems including a hospital information system (HIS), a picture archiving and communication system (PACS), an electronic medical recording system (EMR), and a radiology information system (RIS).

The medical information processing apparatus 2 is a computer that executes an application (hereinafter referred to as a “medical care sharing application”) for sharing information of a target care among the members of the care team. The medical information processing apparatus 2 receives medical service information relating to the target care from the hospital information system 1. In accordance with various commands from the portable devices 3, the medical information processing apparatus 2 executes the processing on the medical care sharing application. The medical information processing apparatus 2 provides the portable device 3 with the input screen data of the medical care sharing application. On the medical care sharing application, the medical service information relating to the target care is shared by the team members of the care team through the portable devices 3. The medical information processing apparatus 2 is also capable of executing an application incorporated in the medical care sharing application for exchanging messages among the team members of the care team (hereinafter referred to as a “chat application”).

The portable device 3 is a computer carried by individual team members of the care team. The portable device 3 here is assumed to be a smartphone, but any mobile computer will do, such as a smartwatch, a notebook PC, and a tablet terminal, into which a medical care sharing application and a chat application can be installed. The portable device 3 includes a processor, an input device, a display device, and a communication device. The portable device 3 serves as an input/output interface of the medical information processing apparatus 2. In one example, the portable device 3 transmits commands to the medical information processing apparatus 2, and displays an input screen provided from the medical information processing apparatus 2 in response to these commands.

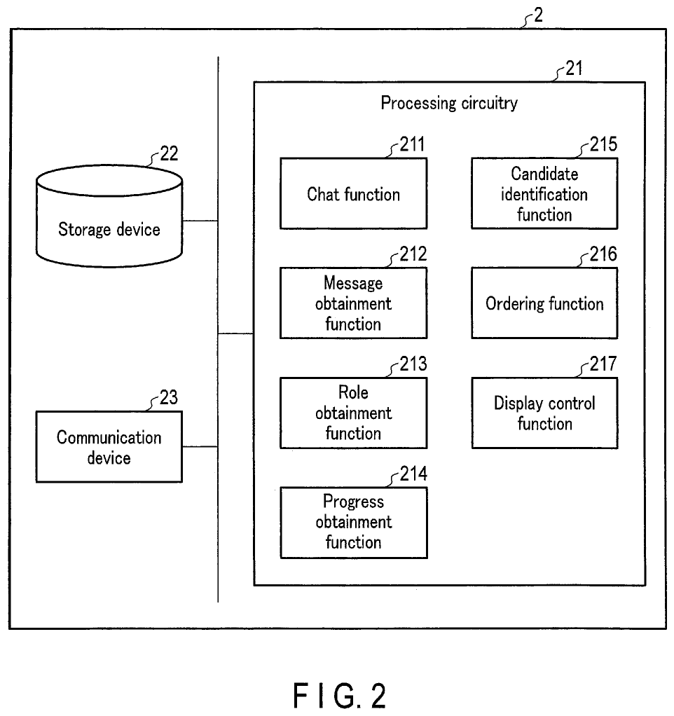

FIG. 2 shows an exemplary configuration of the medical information processing apparatus 2. As illustrated in FIG. 2, the medical information processing apparatus 2 includes a processing circuitry 21, a storage device 22, and a communication device 23. The processing circuitry 21, storage device 22, and communication device 23 are connected to one another via a bus such that signals can be input and output.

The processing circuitry 21 includes a processor such as a central processing unit (CPU). Upon implementation of the medical care sharing application, the processing circuitry 21 realizes a chat function 211, a message obtainment function 212, a role obtainment function 213, a progress obtainment function 214, a candidate identification function 215, an ordering function 216, a display control function 217, and the like. Each of the functions 211 to 217 is not limited to being realized by a single processing circuitry. A plurality of independent processors may be combined into a processing circuitry, and each of the processors may execute a medical information processing program to realize the functions 211 to 217. Furthermore, each of the functions 211 to 217 may be a program designed into a module that constitutes the medical information processing program. These applications and/or programs are stored in the storage device 22. The medical care sharing application and chat application are contained in the medical information processing program.

With the chat function 211, the processing circuitry 21 exchanges messages among the members of the channel for discussing the target care on the chat application. A channel is a field of the chat application in which messages can be exchanged. On the chat application, multiple channels are set up for multiple issues. Multiple members are registered to each channel so that the registered members can exchange messages over this channel. Message exchanging is synonymous with chatting.

With the message obtainment function 212, the processing circuitry 21 obtains a message record, which is a record of messages that have been input by the members of the channel. The message record is stored in the storage device 22.

With the role obtainment function 213, the processing circuitry 21 obtains role information representing the roles of the target members who belongs to the channel. A target member denotes a member to whom later-described candidate messages are presented. A role denotes a classification or authority of this member, examples of which include the department or specialty of the member, such as a neurologist, neurological interventionalist, emergency physician, and neurological radiologist. For instance, the role information may be obtained from the hospital information system 1. Alternatively, the role information may be stored in the storage device 22 and obtained from the storage device 22.

With the progress obtainment function 214, the processing circuitry 21 obtains progress information, which represents the current progress of the target care. The currently achieved progress of the target care may be represented by the current status (step) of the target care in the processes of consultation, diagnosis, and treatment. The current progress of the target care, however, is not limited to the three-status representation of the consultation, diagnosis, and treatment. Each step may be segmented or may be further represented using derived statuses. Examples of the consultation statuses include issuance of an imaging order for diagnostic imaging, start of imaging of an examination image, end of imaging of the examination image, completion of automatic image analysis, issuance of a laboratory examination order, start of the laboratory examination, end of the laboratory examination, and the like. Examples of therapeutic statuses include issuance of a medication order and an end of medication. The progress information may be obtained, for example from the hospital information system 1. Alternatively, the progress information may be stored in the storage device 22 and obtained from the storage device 22.

With the candidate identification function 215, the processing circuitry 21 identifies one or more candidates (hereinafter referred to as “candidate messages”) for a message to be input by a target member, based on the message record obtained with the message obtainment function 212, the role information obtained with the role obtainment function 213, and/or the progress information obtained with the progress obtainment function 214. Each of the one or more candidate messages include a first candidate message corresponding to a first message, which serves as character strings for a conversational sentence, a second candidate message corresponding to a second message, which serves as a medical procedures order, or a third candidate message corresponding to a third message, which serves as a query for information. For instance, the processing circuitry 21 identifies a candidate message for a message to be input by a target member, using a model (hereinafter referred to as a “first model”), based on the role information with the role obtainment function 213 and/or the progress information obtained with the progress obtainment function 214.

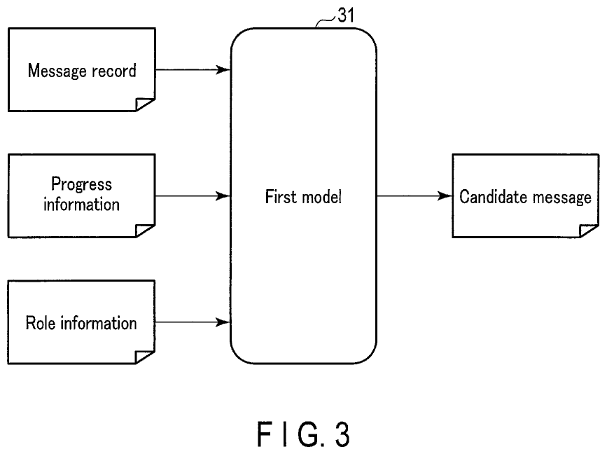

FIG. 3 shows exemplary inputs and outputs of the first model 31. As illustrated in FIG. 3, the first model 31 represents the relationship between (i) the message record, role information, and progress information and (ii) a candidate message. The first model 31 may be a look-up table (LUT) or a database, or may be a machine learning model such as a support vector machine and deep learning model. The first model 31 can be generated based on a set of training samples, which is a set of combinations of input samples including the message record, role information, and progress information and output samples of candidate messages corresponding to the message record, role information, and progress information. The first model 31 according to the present embodiment is assumed to be a deep learning model for outputting a multidimensional vector having a likelihood value for every candidate message as its elements. The likelihood value corresponds to a recommendation level. For instance, the candidate message corresponding to the largest likelihood value among the candidate messages is output as the first candidate, and the candidate message corresponding to the second largest likelihood value is output as the second candidate. The number of candidates to be output can be freely determined.

The inputs of the first model 31 are not limited to the three types, namely the message record, role information, and progress information. The first model 31 may be configured in such a manner that at least one of the message record, role information, and progress information can be input thereto, as needed. The first model 31 may be constituted by a single model, or by multiple models provided by dividing the process from input to output. The first model 31 is stored in the storage device 22.

With the ordering function 216, when the second candidate message is selected by way of the portable device 3, the processing circuitry 21 places an order for a medical treatment corresponding to this candidate. For instance, the processing circuitry 21 issues the order to the HIS or RIS in the hospital information system 1.

With the display control function 217, the processing circuitry 21 displays various types of information on the portable device 3. The processing circuitry 21 may display an input screen of the medical care sharing application or chat application on the portable device 3. In particular, the processing circuitry 21 displays a candidate message identified with the candidate identification function 215 on the input screen of the chat application.

The storage device 22 may include a read-only memory (ROM), a random-access memory (RAM), a hard disk drive (HDD), a solid state drive (SSD), an integrated circuit memory device, and the like, which can store various types of information. In addition to these memory devices, the storage device 22 may be a portable storage medium such as a compact disc (CD), a digital versatile disc (DVD), and a flash memory, or a drive device for reading and writing various types of information from and to a semiconductor memory device or the like. Alternatively, the storage device 22 may be provided in a separate computer connected via a network.

The communication device 23 is an interface for transmitting and receiving various types of information to and from other computers such as the hospital information system 1 and portable devices 3 included in the medical information system 100.

Exemplary processing performed by the medical information processing apparatus 2 according to the present embodiment on the chat application will be described below.

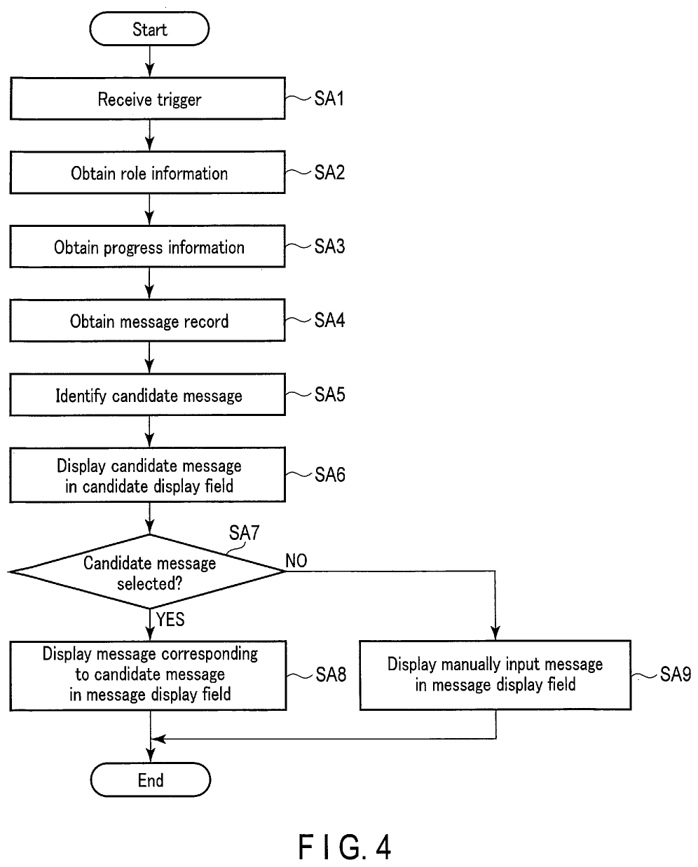

FIG. 4 shows an exemplary procedure of the processing performed by the medical information processing apparatus 2 according to the first embodiment on the chat application. It is assumed here that at the start of step SA1, the processing circuitry 21 has already started the chat application with the chat function 211, for a target channel set up for a target care. Multiple members have been registered for this target channel. The processing of FIG. 4 is performed for each of the members. The “target member” represents a team member for which the processing is performed.

As indicated in FIG. 4, the processing circuitry 21 receives a trigger with the chat function 211 (step SA1). The trigger serves as a cue for offering candidate messages. For instance, a switching moment of the progress status, such as the issuance of an imaging order, a start of the imaging of an examination image, an end of the imaging of the examination image, completion of automatic image analysis, issuance of a laboratory examination order, a start of the laboratory examination, an end of the laboratory examination, issuance of a mediation order, or an end of the medication, may serve as a trigger. In addition, inputting of a message on the chat application may be configured to serve as a trigger.

After step SA1, the processing circuitry 21 obtains the role information of the target member with the role obtainment function 213 (step SA2). After step SA2, the processing circuitry 21 obtains the progress information of the target care with the progress obtainment function 214 (step SA3). After step SA3, the processing circuitry 21 obtains a record of messages relating to the target channel with the message obtainment function 212 (step SA4). The order of steps SA2, SA3 and SA4 is not limited thereto, and may be suitably changed. Alternatively, part of, or all of, steps SA2, SA3, and SA4 may be implemented in parallel.

After step SA4, the processing circuitry 21 identifies one or more candidate messages with the candidate identification function 215, based on the role information obtained at step SA2, the progress information obtained at step SA3, and/or the record of messages obtained at step SA4 (step SA5). At step SA5, the processing circuitry 21 inputs the role information, progress information, and/or record of messages into the first model to identify a candidate message.

Possible combinations of the role information, progress information and record of messages are vast in number. For instance, the processing circuitry 21 may narrow down the candidate messages in accordance with the role information of the target member. In this case, the first model includes a second model, and a third model.

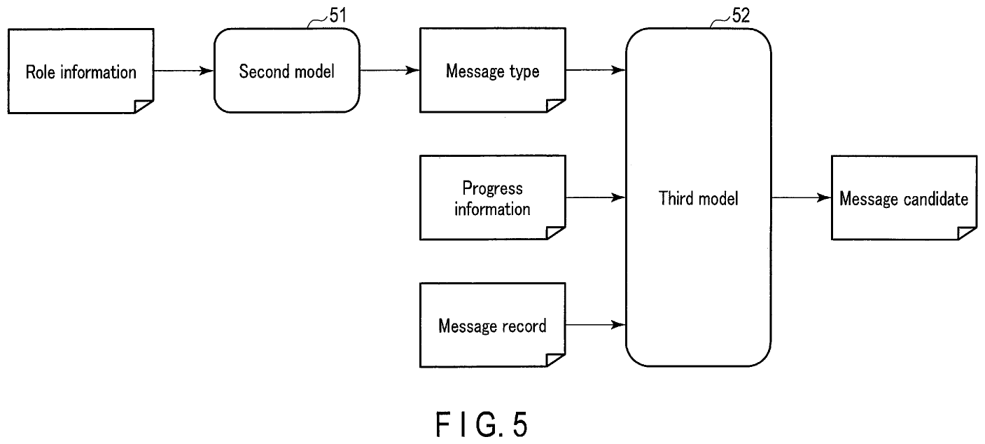

FIG. 5 shows an example of the candidate message identifying process using the second model 51 and third model 52. The second model 51 represents the relationship between the role information of each team member and a type of message that the member is allowed to input. Upon input of the role information, the second model 51 outputs a type of message corresponding to the role information. The second model 51 may be an LUT or database, or may be a machine learning model such as a support vector machine or deep learning model. The second model 51 is generated based on a set of training samples, which are combinations of input samples including role information and output samples including types of messages corresponding to the role information. The second model 51 is stored in the storage device 22.

As illustrated in FIG. 5, the third model 52 represents the relationship between (i) the message types, progress information, and record of messages and (ii) candidate messages. The record of messages may be the messages as-is that have been input to the target channel, or the current state of the target care that can be estimated from these messages. Upon an input of the message type, progress information, and record of messages, the third model 52 outputs a candidate message. The third model 52 may be an LUT or database, or may be a machine learning model such as a support vector machine or deep learning model. The third model 52 is generated based on a set of training samples, which are combinations of input samples including message types, progress information, and records of messages, and output samples including candidate messages. The third model 52 is assumed to be a deep learning model configured to output a multidimensional vector having a likelihood value for every candidate message as its elements. The third model 52 is stored in the storage device 22.

Using the second model 51 and third model 52, a candidate message is identified. Specifically, the processing circuitry 21 first identifies the message type that the target member is allowed to input, based on the role information of the target member and the second model 51. Thereafter, the processing circuitry 21 identifies one or more candidate messages, based on the message type, progress information, record of messages, and third model 52. According to this processing procedure, the type of candidate messages is narrowed based on the role information of the target member, and a candidate message is identified from the narrowed candidate messages, based on the progress information and record of messages.

After step SA5, the processing circuitry 21 displays, with the display control function 217, candidate messages identified at step SA5 in a candidate display field (step SA6). At step SA6, the processing circuitry 21 displays the candidate messages in the candidate display field included in the input screen of the chat application on the portable device 3 of the target member. Here, the processing circuitry 21 displays one or more candidate messages that have been identified, by adopting visual effects corresponding to the function of the candidate message. In particular, candidate messages that function as character strings for a conversational sentence, candidate messages that function as orders for medical procedures, and candidate messages that function as a query for information may be presented with different visual effects, such as different colors, patterns, geometries, and sizes.

After step SA6, the processing circuitry 21 determines, with the display control function 217, whether or not a candidate message displayed at step SA6 is selected (step SA7).

If it is determined that the candidate message is selected (yes at step SA7), the processing circuitry 21 displays a message corresponding to the selected candidate message in the message display field on the input screen (step SA8). At step SA8, the processing circuitry 21 displays a message in the message display field included in the input screen on the chat application displayed on the portable device 3 of the target member.

If it is determined that the candidate message is not selected (no at step SA7), the processing circuitry 21 displays a manually input message on the message display field (step SA9).

After step SA8 or SA9, the processing circuitry 21 terminates the processing of FIG. 4. Thereafter, the processing circuitry 21 waits for a trigger to be input, and upon receipt of a trigger, the processing circuitry 21 executes the operations at steps SA1 through SA8 or SA9.

The obtainment of the role information at step SA2, obtainment of the progress information at step SA3, and obtainment of the record of messages at step SA4 do not always need to be executed every time a trigger is received at step SA1. If the processing circuitry 21 has already obtained the information, the obtained information can be used, without the need to execute the operations of steps SA2 to SA4.

Next, examples of the processing on the chat application will be described.

### Example 1

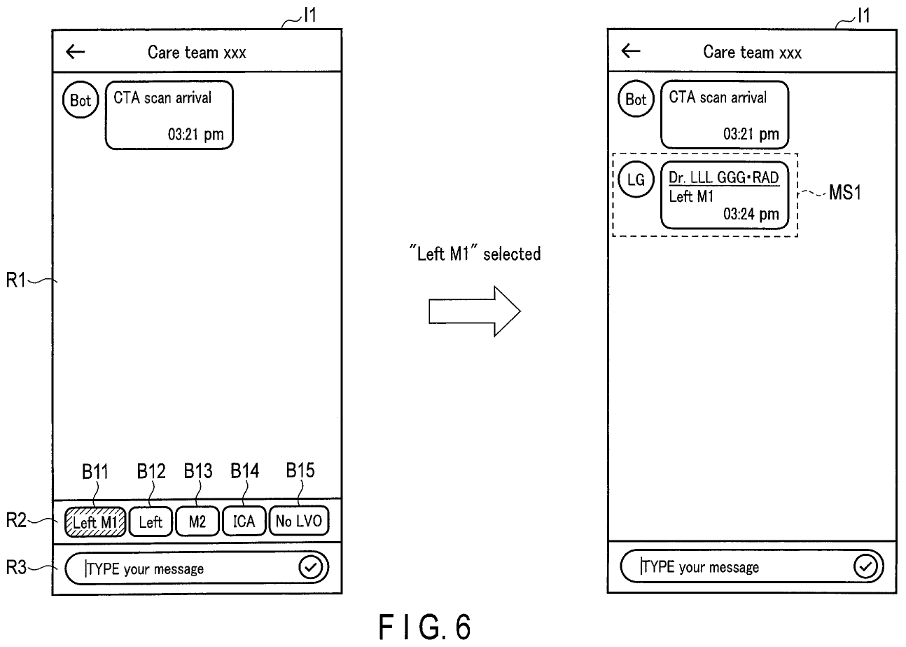

FIG. 6 shows a transition of an input screen I1 on the chat application in Example 1. The input screen I1 is displayed on the portable device 3 by the processing circuitry 21 with the display control function 217. As illustrated on the left side of FIG. 6, the input screen I1 contains a message display field R1, a candidate display field R2, and a manual input field R3. At the top portion of the input screen I1, the name of the care team for the target care, “xxx”, may be indicated.

Chat messages that are input by the members registered for the target channel are listed chronologically in the message display field R1. Each message is constituted by an icon representing the sender of the message and a display frame for displaying character strings that represent the content of the message. In the display frame, the full name and role information of the message sender and the time and date of the message sent are displayed in addition to the character strings representing the content of the message. As an icon, the sender's initials may be presented. The icon on the left side of FIG. 6 shows “Bot”, which indicates that the sender of the message is the robot installed in the chat application. This robot can be realized by the processing circuitry 21 with the chat function 211. For instance, messages relating to the current progress of the target care, such as messages that report the completion of medical imaging and the completion of automatic image analysis by the hospital information system 1, are automatically input. On the left side of FIG. 6, “CTA scan arrival” is displayed in relation to the completion of imaging a contrast enhanced CT image to report the arrival of the contrast enhanced CT image at the hospital information system 1.

In the candidate display field R2, one or more candidate messages identified with the candidate identification function 215 are displayed. The candidate messages are displayed as graphical user interface (GUI) buttons B11, B12, B13, B14, and B15, each representing a character string that indicates a candidate message. When a candidate message is selected by way of the portable device 3, a message corresponding to the selected candidate message is displayed in the message display field R1. For instance, when B11 for a candidate message “left M1” is selected, a message MS1 “left M1” corresponding to this candidate message is displayed in the message display field R1. To increase the visibility of the message display field R1, it is preferable that the candidate display field R2 be deleted from the input screen I1 after a candidate message is selected.

The manual input field R3 is a GUI component for manual input of a message by a member into the portable device 3. For instance, if there is no suitable candidate message among the ones displayed in the candidate display field R2, a message is manually input into the manual input field R3. The manually input message is displayed in the message display field R1.

The candidate message according to Example 1 is a candidate for a message that functions as a character string for a conversational sentence. If the role information of the target member is a radiologist, and if the progress information shows the completion of the image analysis, the processing circuitry 21 according to Example 1 displays, as a candidate message, a GUI button (hereinafter referred to as a “chat button”) for inputting the image diagnosing result, with the candidate identification function 215.

In the example of FIG. 6, the name of the target member is “LLL GGG”, whose initials are “LG”. The role information of this target member is a radiologist (RAD), and the progress information indicates the completion of contrast enhanced CT imaging. Upon the completion of the contrast enhanced CT imaging, the hospital information system 1 conducts an automatic image analysis upon the contrast enhanced CT images. It is assumed that the contrast enhanced CT imaging has been conducted upon a patient who is suspected of having cerebral infarction, and an analysis regarding the presence/absence of cerebral infarction has been conducted upon the contrast enhanced CT images. The information of this patient suspected of having cerebral infarction is managed and stored by the hospital information system 1 as medical service information on the electronic chart, examination order, and the like. The image analysis estimates that the patient has an acute-phase cerebral infarction caused by large vessel occlusion (LVO), with clogging in the M1 vessel of the left brain. The result of the image analysis is managed and stored by the hospital information system 1 as the medical service information on the electronic chart or the like.

Under the above circumstances, the processing circuitry 21 narrows down the candidate messages to character strings that represent the image diagnosing results relating to contrast enhanced CT images, based on the target member's role information “radiologist” and the progress information “completion of contrast enhanced CT imaging”. Furthermore, based on the medical service information demonstrating that the patient is suspected of having cerebral infarction and that LVO has been caused in the M1 vessel of the left brain, the processing circuitry 21 identifies, as a first candidate message, a character string “left M1”, which indicates an image diagnosing result that the LVO is suspected in the M1 vessel of the left brain. Based on this medical service information, the processing circuitry 21 may identify other vessels having inclusion relationship or proximity relationship with the M1 vessel of the left brain from the aspect of anatomy, as auxiliary candidate messages. In the example of FIG. 6, a character string “left” representing a vessel in the left brain, a character string “M2” representing the M2 vessel, and a character string “ICA” representing the internal carotid artery are identified as auxiliary candidate messages. In addition, a character string “No LVO” representing that the LVO is not suspected may be identified as an auxiliary candidate message.

Chat buttons B11, B12, B13, B14, and B15 showing these candidate messages are displayed in the display field R2. The candidate messages are displayed from the left to the right in descending order of recommendation levels. The chat button B11 for “left M1”, which is the first candidate, is shaded, while the chat buttons B12, B13, B14, and B15 respectively for “left”, “M2”, “ICA”, and “No LVO”, which are other candidate messages, are unshaded. With the candidate messages displayed with different visual effects depending on their recommendation levels, the team members can be assisted in selection of a candidate message.

If the chat button B11 for the candidate message “left M1” is selected, the processing circuitry 21 displays, as a message from the target member “Dr. LLL GGG”, a message MS1 “left M1” corresponding to the candidate message “left M1” in the message display field R1, as illustrated on the right side of FIG. 6. The message MS1 “left M1” may be displayed, for example in non-bold black, to produce visual effects for showing that the message type is a character string.

According to Example 1, candidates for a character string to be input by a target member are displayed as candidate messages in accordance with the role of the target member and progress of the target care, as a result of which the target member's cumbersome task of inputting a character string can be reduced.

### Example 2

The candidate message according to Example 2 is a candidate for a message that functions as a medical procedures order. The medical treatment according to Example 2 includes any treatment managed by the hospital information system 1, such as surgeries, medication, medical imaging, analysis of medical images, and reading of medical images. With the candidate identification function 215, if the role information of the target member indicates a physician specialized in the symptom suspected in the target care, the processing circuitry 21 according to Example 2 displays a GUI button (hereinafter referred to as an “order button”) as a candidate message to issue a medical procedures order for dealing with the symptom.

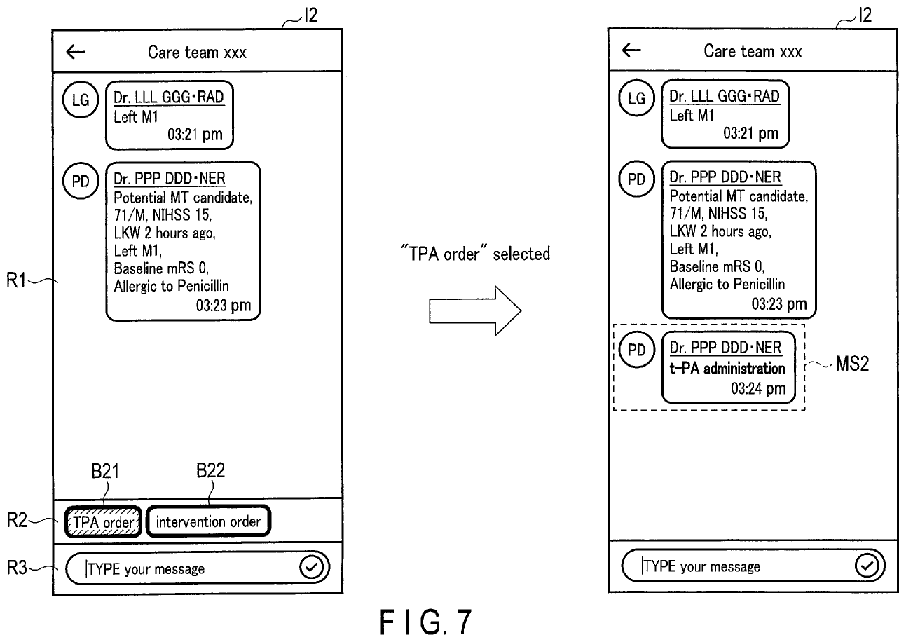

FIG. 7 shows a transition of an input screen I2 on the chat application in Example 2. The input screen I2 has a layout similar to that of the input screen I1 in FIG. 5. The input screen I2 of FIG. 7 is a continuation of the input screen I1 of FIG. 5. The name of the target member according to Example 2 is “PPP DDD”, whose initials are “PD” and whose role information is “neurological physician (NER)”. As illustrated in FIG. 7, the message display field R1 presents a character string “left M1” that has been input by the radiologist member “LG” to indicate the image diagnosing result, and also a character string input by the target member “PD” to show the current conditions relating to the target care. In particular, the NIHSS score being 15, the onset time or in other words the last known well (LKW) time being two hours ago, and the target patient being allergic to penicillin are entered. These messages are stored in the storage device 22 as a record of messages. The onset time being two hours ago indicates that the administration of t-PA is clinically possible.

Under such circumstances, the processing circuitry 21 uses the second model to narrow down the candidate messages to neurological treatment orders, based on the role information “neurological physician” of the target member. The processing circuitry 21 analyzes the record of messages and determines that the neurological physician has diagnosed the patient with LVO. The processing circuitry 21 further analyzes the record of messages to identify the onset time as two hours ago. The processing circuitry 21 uses the third model to identify, as the first candidate message, “TPA order” to issue a t-PA administration order, based on the message type “neurological treatment order”, the progress information “completion of contrast enhanced CT imaging”, and the record of messages “neurological physician's diagnosis being LVO” and “onset time being two hours ago”. As other candidates, the processing circuitry 21 may identify the order for surgery on the LVO, “intervention order”, as an auxiliary candidate message to issue a medical procedure order other than the t-PA administration.

The order buttons B21 and B22 for the above candidate messages are displayed in the candidate display field R2. B21 and B22 for the candidate messages are displayed from the left to the right in descending order of recommendation levels. B21 for the message “TPA order” is shaded as the first candidate, while B22 for the other candidate message “intervention order” is unshaded. In order to distinguish the types of candidate messages, or in other words to mutually distinguish character strings and orders, candidate messages are displayed in a visually distinguishable manner. For instance, in the example of FIG. 7, the order buttons B21 and B22 are enclosed with a heavy line. In FIG. 6, the chat buttons B11, B12, B13, B14, and B15 are enclosed in a thin line. This allows the target member to ascertain at a glance that the displayed candidate message is an order due to the visual effects of the GUI buttons.

If the order button B21 for the candidate message “TPA order” is selected, the processing circuitry 21 displays the message MS2 “TPA order” corresponding to the candidate message “TPA order”, as a message from the target member “Dr. PPP DDD”, in the message display field R1, as illustrated on the right side of FIG. 7. The message MS2 “TPA order” is displayed preferably in a font corresponding to the message type being an order, for example in bold. Furthermore, if the candidate message “TPA order” is selected, the processing circuitry 21 automatically issues, with the ordering function 216, the t-PA administration order to the hospital information system 1, which is the order recipient. The processing circuitry 21 may display an ordering button to prompt the target member to provide the final instructions for the t-PA administration order. In this case, upon the depression of the ordering button, the processing circuitry 21 issues an order for t-PA administration.

In another example, the processing circuitry 21 may display the authorizer of the t-PA administration order on the input screen, together with the ordering button. When the ordering button is pressed, the processing circuitry 21 displays two GUI buttons on the display screen of the authorizer's portable device 3, one for approving the order (hereinafter referred to as an “approval button”) and the other for disapproving the order (hereinafter referred to as a “denial button”). Upon depression of the approval button, the processing circuitry 21 automatically issues the t-PA administration order to the hospital information system 1, which is the order recipient. Upon depression of the denial button, the processing circuitry 21 may dismiss the t-PA administration order and display, on the target member's input screen, text indicating that the order has been dismissed by the authorizer.

According to Example 2, candidates for the order to be issued by the target member are displayed as candidate messages, as a result of which the target member's cumbersome task of inputting an order can be reduced. Furthermore, a message corresponding to the order is displayed in the message display field, which allows the team members to readily share the issuance of the order.

As an example of an order issued, a t-PA administration order is discussed above. The order is not limited thereto, however, and a surgery order, a medical imaging order, a medical image reading order, and the like may be issued.

### Example 3

The candidate message according to Example 3 is a candidate for a message that functions as a query requesting the onset time. In Example 3, if the role information of the target member indicates the physician specialized in the symptom suspected in the target care, and if no message relating to the onset time of the symptom is included in the record of messages, the processing circuitry 21 displays, as a candidate message, a GUI button for asking the onset time (hereinafter referred to as an “onset time query button”).

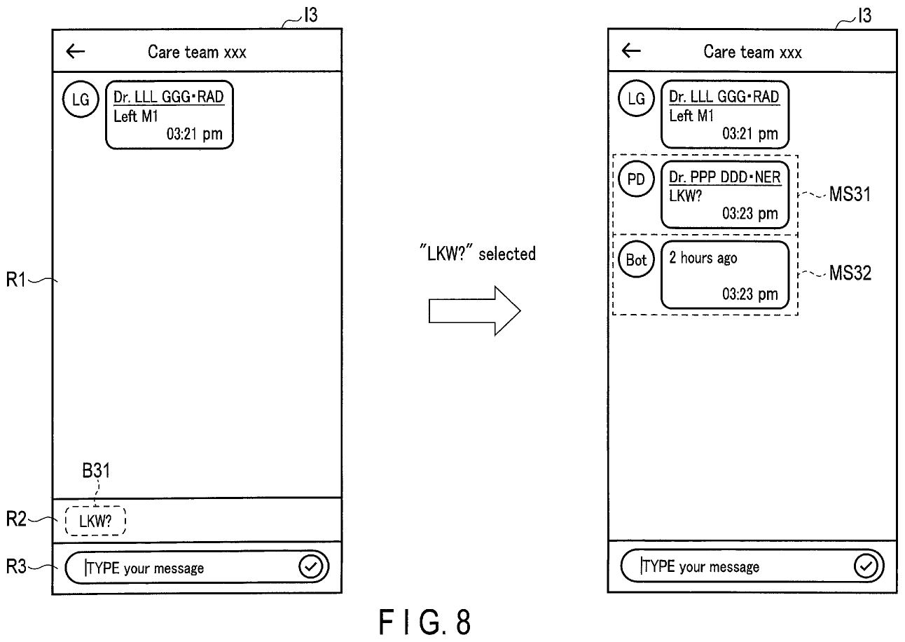

FIG. 8 shows a transition of an input screen I3 on the chat application in Example 3. The input screen I3 has a layout similar to that of the input screen I1 in FIG. 5. The input screen I3 of FIG. 8 is a continuation of the input screen I1 of FIG. 5. As illustrated in FIG. 8, a character string “left M1” indicating the image diagnosing result is entered by the member “LG”, who is a radiologist, to the display field R1. In Example 3, it is assumed that the target member is “PD”, who is a neurological physician. Due to the radiologist's diagnosis of LVO, it is necessary for the target member to find the onset time in order to determine whether or not to proceed with t-PA administration.

Under such circumstances, the processing circuitry 21 uses the second model, based on the role information “neurological physician” of the target member, to narrow down the candidate messages to neurological treatment orders. The processing circuitry 21 further analyzes the record of messages and determines that the neurological physician has diagnosed the patient with LVO. The processing circuitry 21 also analyzes the record of messages in the target channel to identify that there is no message relating to the onset time. The processing circuitry 21 uses the third model to identify, as the first candidate, a candidate message “LKW?” to request the onset time, based on the message type “neurological treatment order”, the progress information “completion of the contrast enhanced CT imaging”, and the record of messages indicating “neurological physician's diagnosis being LVO” and “no message found relating to the onset time”.

The onset time query button B31 for the candidate message “LKW?” is displayed in the display field R2. In order to distinguish the types of candidate messages, or in other words to distinguish character strings, orders, and queries from each other, the candidate messages are displayed in a visually distinguishable manner. For instance, in the example of FIG. 8, the onset time query button B31 is enclosed in a dashed line. In this manner, the user can ascertain at a glance that the displayed candidate message is a query.

If the onset time query button B31 for the candidate message “LKW?” is selected, the processing circuitry 21 displays in the display field R1 a message MS31 “LKW?” corresponding to the candidate message “LKW?” as a message from the target member “Dr. PPP DDD”, as illustrated on the right side of FIG. 8. Preferably, the message MS31 “LKW?” may be displayed with the same visual effects as those for character strings.

Upon display of the message “LKW?”, the processing circuitry 21 analyzes the medical service information stored in the hospital information system 1 to extract or predict the onset time of the target patient. If the onset time is extracted or predicted, the processing circuitry 21 displays the message MS32 indicating the onset time as a message from the sender “Bot” in the message display field R2. For instance, if the onset time is two hours ago, the message MS32 “2 hours ago” is displayed.

If the onset time is not extracted or predicted from the hospital information system 1, the processing circuitry 21 may display in the display field R1 a message requesting other members to enter the onset time. A person on the team who sees this message will input a character string representing the onset time as a message on the portable device 3.

According to Example 3, a candidate GUI button for requesting information that may possibly be sought by the target member is displayed as a candidate message, as a result of which the target member's cumbersome task of inputting text for the query can be reduced. Furthermore, upon the depression of a GUI button, a response to the query is automatically found or predicted from the hospital information system 1 and is displayed in the message display field, which can reduce the cumbersome task of obtaining a response.

### Example 4

The candidate message according to Example 4 is a candidate for a message that functions as a query requesting treatment content information relating to the target care. If the target member is a newly invited member to the target channel, the processing circuitry 21 according to Example 4 displays as a candidate message a GUI button (hereinafter referred to as a “treatment content query button”) for requesting treatment content information relating to the details of the target care.

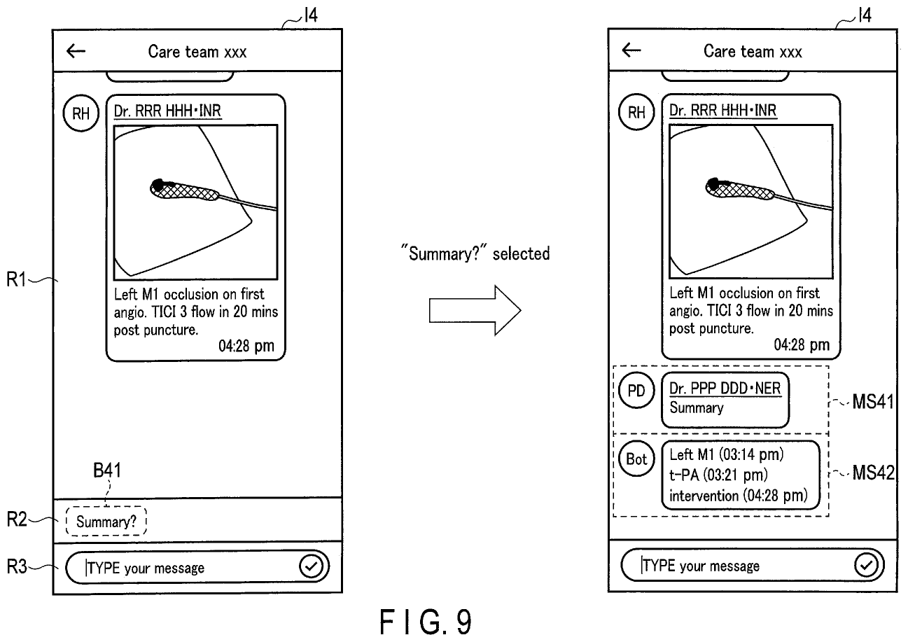

FIG. 9 shows a transition of an input screen I4 on the chat application in Example 4. The input screen I4 has a layout similar to that of the input screen I1 in FIG. 5. It is assumed that the target member is the neurological physician “PD”. This target member has been newly invited to a target channel relating to a target care, and has been asked by a neurological interventionalist “RH” for remarks on the target care. Since this target channel was opened long before, a number of messages have been entered in the message display field R1 of the input screen I4.

If the target member is someone newly invited to the target channel and if the record of messages of the target channel satisfies a treatment content query condition, the processing circuitry 21 determines to display the treatment content query button. The treatment content query condition indicates a situation in which a large number of messages are included in the message record. In particular, the treatment content query condition may be determined to be the number of messages in the record of messages being equal to or larger than a threshold value, or to a certain length or more of time elapsed since the opening of the target channel.

If the treatment content query condition is satisfied, the processing circuitry 21 displays a treatment content query button B41 that represents “Summary?” in the candidate display field R2, as illustrated in FIG. 9. The treatment content query button B41, which is a candidate query message, is enclosed in a dashed line.

If the treatment content query button B41 for the candidate message “Summary?” is selected, the processing circuitry 21 displays the message MS41 “Summary” corresponding to the candidate message “Summary?” in the message display field R1, as a message from the target member “Dr. PPP DDD”, as illustrated on the right side of FIG. 9. Preferably, the message MS41 “Summary” is displayed with the same visual effects as those for character strings.

Upon display of the message MS41 “Summary”, the processing circuitry 21 reads the record of messages of the target channel stored in the storage device 22, and creates a summary text for the read-out message record. The method for creating a summary text is not particularly limited. For example, based on the role information of the target member, progress information of the target care, and/or record of messages, the processing circuitry 21 predicts the contents that the target member may currently desire to have, extracts the messages relating to the predicted contents from the record of messages, and creates a summary text by combining the extracted messages. The summary text may preferably include, in addition to the extracted messages, the times and dates of these messages sent.

If a summary text is created, the processing circuitry 21 displays a message MS42 including this summary text as a message from the sender “Bot” in the message display field R1. In the situations of Example 4, the role information of the target member is a neurological physician, the progress information indicates the completion of a surgery, and reporting of the surgery result is included in the record of messages. The contents that the target member possibly desires to have at the moment therefore can be estimated as the image diagnosing result and details of the medical treatment provided. Thus, the summary text of, for example, “Left M1 (03:14 pm), t-PA (03:21 pm), intervention (04:28 pm)” is created and displayed.

According to Example 4, a treatment content query button is displayed for a target member who is newly invited to a certain channel, as a result of which the target member's cumbersome tasks of grasping the contents of the record of messages can be reduced. For other members also, the task of explaining the record of messages to the target member can be omitted.

### Example 5

The candidate message according to Example 5 is a candidate for a message that functions as a query requesting a task assigned to the target member. If the alert condition is satisfied while the task assigned to the target member in the target channel is incomplete, the processing circuitry 21 according to Example 5 displays a button for requesting the details of the task (hereinafter referred to as a “task query button”), as a candidate message.

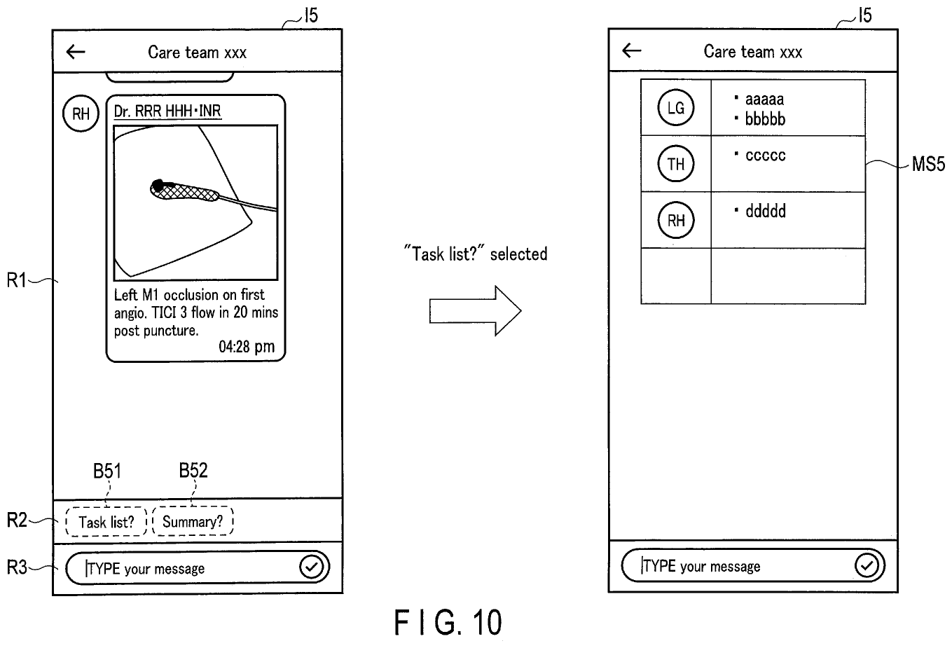

FIG. 10 shows a transition of an input screen I5 on the chat application in Example 5. The input screen I5 has a layout similar to that of the input screen I1 in FIG. 6. The target member in Example 5 is in the same situation as in Example 4. That is, the target member is the neurological physician “PD”, who has been newly invited to a target channel relating to a target care. The target member has been asked by the neurological interventionalist “RH” for remarks on the target care. Since this target channel was opened long time before, a number of messages have been entered in the message display field R1 of the input screen I5. Thus, a treatment content query button B52 is displayed in the candidate display field R2, as described in Example 4.

The processing circuitry 21 according to Example 5 determines that a task query button will be displayed if the task assigned to a target member is incomplete and the alert condition is satisfied in the target channel. The alert condition indicates a condition in which in a certain target channel, the task assigned to a target member appears to be incomplete. In particular, the alert condition may be set to a task assigned to a target member being incomplete, to a certain length of time being elapsed with the task being incomplete, or to the number of incomplete tasks exceeding a threshold value.

The processing circuitry 21 determines whether a task is assigned to the target member and whether the task is incomplete, based on the role information of the target member, the progress information of the target care, and/or the record of messages of the target channel. For instance, if the record of messages includes a message to the target member, directly requesting a task such as “Mr. PPP DDD, please do xx”, the processing circuitry 21 determines that a task has been assigned to the target member. Alternatively, based on the role information of the target member, progress information of the target care, and/or record of messages in the target channel, the processing circuitry 21 may estimate the current situation in which the target member is placed, identify a specific task corresponding to the current situation, and determine that this task has been assigned to the target member.

The task assigned to the target member can be determined as being incomplete if the record of messages includes a message that directly indicates the task is incomplete, for example “xx not yet completed”, or if the record of messages lacks a message that directly indicates the completion of the task, for example “xx completed”. Alternatively, based on the role information of the target member, progress information of the target care, and/or the record of messages in the target channel, the processing circuitry 21 may estimate the current situation in which the target member is placed, and determine that the task is incomplete if it is estimated based on this current situation that the target member has not finished the task.

If the alert condition is satisfied, the processing circuitry 21 displays a task query button B51 that represents “Task list?” in the candidate display field R2, as illustrated on the left side of FIG. 10. The task query button B51, which is a candidate query message, is enclosed in a dashed line.

If the task query button B51 for the candidate message “Task list?” is selected, the processing circuitry 21 displays a list MS5 of incomplete tasks (hereinafter referred to as a “task list”) assigned to the target members in the message display field R1, as illustrated on the right side of FIG. 10. In the task list MS5, the requesters of incomplete tasks and the descriptions of the tasks are associated and listed in the form of a table. For instance, in the example of FIG. 10, the target member “PD” has received two tasks, namely tasks “aaaaa” and “bbbbb” from the requester “LG”. By checking the task list MS5, the target member can readily ascertain incomplete tasks assigned to her/himself. The display of the task list MS5 is not limited to the message display field R1. The window displaying the task list MS5 may be displayed in a manner overlapping on the input screen I5.

According to Example 5, a task query button is displayed for the target member, as a result of which the target member's cumbersome tasks and time in ascertaining the incomplete tasks can be reduced. Furthermore, it is expected that the target member can easily ascertain the tasks, and therefore can smoothly carry out these tasks.

### Modification Examples

Several modification examples of the present embodiment will be described.

### Modification Example 1

In a situation in which comments from a non-member specialist are required, the processing circuitry 21 may display a GUI button (hereinafter referred to as an “invitation button”) on the input screen in order to invite this specialist to the target channel. The specific processing procedure is described below. First, the processing circuitry 21 estimates the current situation of the target care based on the progress information of the target care and the record of messages of the target channel. The processing circuitry 21 determines whether there is currently a shortage of any role in the target channel, based on the current situation and role information of the members registered in the target channel. If there is a shortage of a role in the target channel, it means that an opinion from a non-member specialist is needed.

If it is determined that there is a shortage of a role in the target channel, the processing circuitry 21 identifies a person (invitee) suitable for this role from a human resources registry or the like managed by the hospital information system 1, and displays an invitation button to invite this invitee. Upon depression of the invitation button on the portable device 3, the processing circuitry 21 displays a notification to invite the invitee to the target channel, on the invitee's input screen of the chat application. According to the modification example 1, a specialist can be smoothly invited onto the channel.

### Modification Example 2

According to modification example 1, an invitee is invited to the target channel. In the absence of a response from the invitee, however, the processing circuitry 21 may display a GUI button (hereinafter referred to as a “phone call button”) on the input screen so as to call the invitee. In particular, the processing circuitry 21 identifies the phone number of the invitee from the human resources registry or the like managed by the hospital information system 1, and displays the phone number together with the phone call button for calling the invitee. Upon depression of the phone call button on the portable device 3, the phone line will be connected between this portable device 3 and the portable device 3 of the invitee. According to the modification example 2, a phone call can be easily made to a specialist.

### Modification Example 3

In a situation where comments from a non-member specialist are required, the processing circuitry 21 may display a phone call button on the input screen to call the specialist. The method for estimating the situation where a specialist's comments are required is the same as the modification example 1. If it is determined that there is a shortage of a role in the target channel, the processing circuitry 21 identifies a person (invitee) suitable for this role from a human resources registry or the like managed by the hospital information system 1, and displays a phone call button for calling the invitee together with his/her phone number. Upon depression of the phone call button on the portable device 3, the phone line will be connected between this portable device 3 and the portable device 3 of the invitee. According to the modification example 3, a phone call can be easily made to a specialist.

### Modification Example 4

In the implementation of the chat application, when an examination image is received by the hospital information system 1, the processing circuitry 21 may display a GUI button (hereinafter referred to as a “first transition button”) on the input screen of the radiologist to switch to the display screen of this examination image. For instance, if a CT examination image is received by the hospital information system 1, the hospital information system 1 notifies the medical information processing apparatus 2 of the arrival of the CT examination image. Upon receipt of this notification, the processing circuitry 21 identifies a radiologist from among the members registered for the target channel relating to this CT examination image. Then, the processing circuitry 21 displays the first transition button on the input screen of this radiologist. Upon depression of the first transition button on the portable device 3 of the member who is a radiologist, the CT examination image is displayed on this radiologist's portable device 3. According to the modification example 4, examination images can be swiftly and easily viewed.

### Modification Example 5

In the implementation of the chat application, when the automatic image analysis of an examination image is completed, the processing circuitry 21 may display a GUI button (hereinafter referred to as a “second transition button”) on the radiologist's input screen to switch to the display screen showing the result of the automatic image analysis. For instance, if a CT examination image is received by the hospital information system 1, an automatic image analysis is conducted upon the CT examination image by the hospital information system 1 or the like. Upon completion of the automatic image analysis, the hospital information system 1 notifies the medical information processing apparatus 2 of the completion of the automatic image analysis. Upon receipt of this notification, the processing circuitry 21 identifies a radiologist from among the members registered for the target channel relating to the CT examination image. Then, the processing circuitry 21 displays the second transition button on the input screen of this radiologist. Upon depression of the second transition button on the radiologist's portable device 3, the analysis result of the CT examination image is displayed on this radiologist's portable device 3. According to the modification example 5, the result of the automatic image analysis can be swiftly and easily viewed.

### Modification Example 6

In the implementation of the chat application, a diagnosis-related important character string to be entered into the electronic chart may be input as a message. If a diagnosis-related important character string is input as a message, the processing circuitry 21 may display on the input screen a GUI button (hereinafter referred to as an “attachment button”) to instruct the attachment of this message to the electronic chart. To determine whether or not the character string is important in the diagnosis, a technique with an application of machine learning such as word2vec may be adopted. Upon depression of the attachment button on the portable device 3, the processing circuitry 21 attaches a message corresponding to the diagnosis-related important character string to the appropriate section on the electronic chart of the target care. According to the modification example 6, a cumbersome task of inputting information into an electronic chart can be reduced.

It is not essential, however, to display an attachment button. For instance, if a message displayed in the message display field is selected on the portable device 3, the processing circuitry 21 may attach the selected message to the electronic chart. The message selecting method is not particularly limited, and a long press or double-clicking may be intuitive and preferable.

### Second Embodiment

A medical information processing apparatus according to the second embodiment identifies a candidate message based on the medical service information of the target care in addition to the record of messages, role information, and/or progress information. The medical information processing apparatus according to the second embodiment will be explained. In the explanation below, structural elements having substantially the same operations as in the first embodiment will be denoted by the same reference symbols, and the same explanation will be given only where necessary.

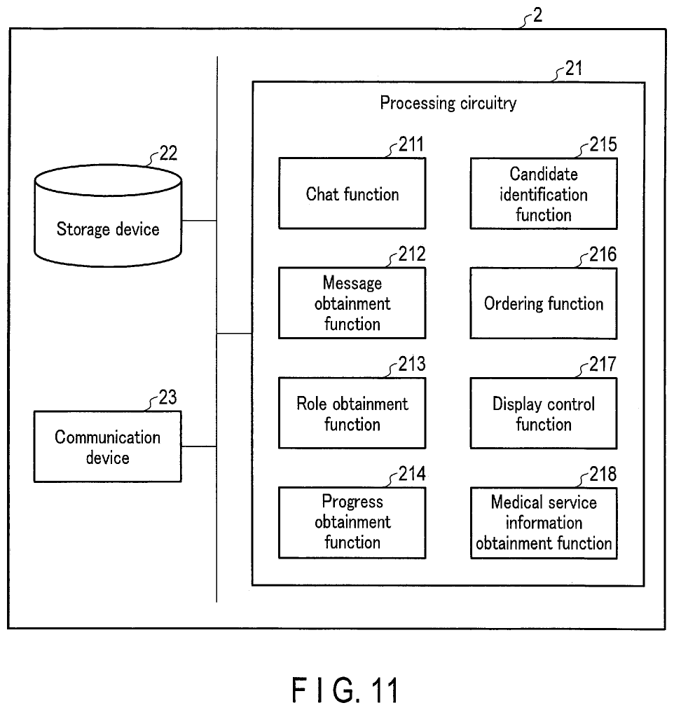

FIG. 11 shows an exemplary configuration of the medical information processing apparatus 2 according to the second embodiment. As illustrated in FIG. 11, in addition to the chat function 211, message obtainment function 212, role obtainment function 213, progress obtainment function 214, candidate identification function 215, ordering function 216, and display control function 217, the processing circuitry 21 of the medical information processing apparatus 2 realizes a medical service information obtainment function 218. The functions 211 to 218 may or may not be realized by a single processing circuitry. A plurality of independent processors may be combined into a processing circuitry, and each of the processors may execute a medical information processing program to realize the functions 211 to 218. Furthermore, each of the functions 211 to 218 may be a program designed into a module that constitutes the medical information processing program. These applications and/or programs are stored in the storage device 22. The medical care sharing application and chat application are contained in the medical information processing program.

With the medical service information obtainment function 218, the processing circuitry 21 obtains medical service information of the target care from the hospital information system 1. The medical service information is generated on various types of computer systems that constitute the hospital information system 1. In particular, the hospital information system 1 may include an electronic medical recording system, an examination reservation system, a PACS, an image processing system, a reporting system, a vitals monitoring system, an electrocardiographic system, and/or a medication information system. The electronic medical recording system generates an electronic chart including the patient's basic information, clinical history, and current medical conditions, as medical service information. The examination reservation system issues orders for various examinations, such as an imaging examination, a blood test, and a medication, as the medical service information. The PACS conducts an imaging examination in accordance with the imaging examination order, and generates a medical image as the medical service information. The image processing system analyzes the presence or absence of any sign on the medical image and generates an image analysis result as the medical service information. The reporting system generates a radiologist's reading report of the medical image as the medical service information. The vitals monitoring system generates vital information such as the patient's heart rate, respiration rate, blood pressure, and oxygen saturation, as the medical service information. The electrocardiographic system generates the patient's electrocardiogram or the electrocardiographic result as the medical service information. Each of these systems may be constituted by a single computer, or a computer network system having two or more mutually communicable computers.

With the candidate identification function 215, the processing circuitry 21 identifies one or more candidate messages, based on the medical service information in addition to the record of messages, role information, and/or progress information.

With the display control function 217, the processing circuitry 21 displays various types of information on the portable device 3. For instance, the processing circuitry 21 displays the candidate messages identified with the candidate identification function 215, on the input screen of the chat application. The processing circuitry 21 may further display the medical service information obtained with the medical service information obtainment function 218.

Exemplary processing performed by the medical information processing apparatus 2 according to the present embodiment on the chat application will be described below.

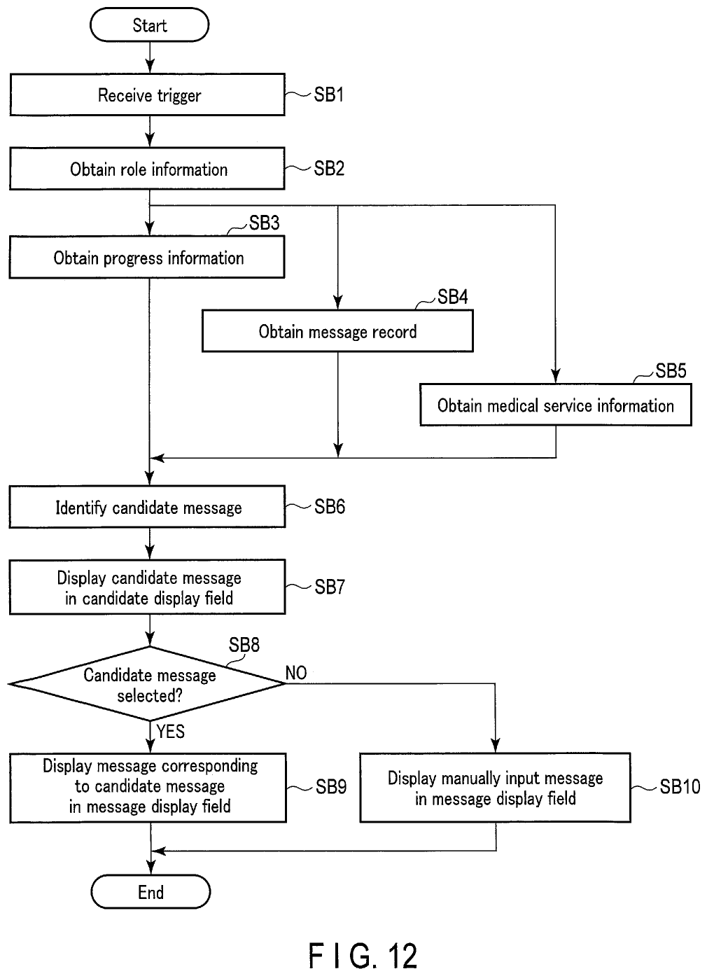

FIG. 12 shows an exemplary processing procedure of the medical information processing apparatus 2 according to the second embodiment on a chat application. It is assumed here that at the start of step SB1, the processing circuitry 21 has already started the chat application with the chat function 211, for the target channel set up for this target care. Multiple members have been registered for this target channel. The processing of FIG. 12 is performed for each of the members.

As indicated in FIG. 12, the processing circuitry 21 receives a trigger with the chat function 211 (step SB1). After step SB1, the processing circuitry 21 obtains the role information of the target member with the role obtainment function 213 (step SB2). The operations at steps SB1 and SB2 are the same as the operations at steps SA1 and SA2, respectively.

After step SB2, the processing circuitry 21 obtains the progress information of the target care with the progress obtainment function 214 (step SB3), obtains the record of messages relating to the target channel with the message obtainment function 212 (step SB4), and obtains the medical service information of the target care with the medical service information obtainment function 218 (step SB5). The order of steps SB3, SB4, and SB5 is not limited thereto, and part or all of the steps may be serially implemented.

After steps SB3, SB4 and SB5, the processing circuitry 21 identifies one or more candidate messages with the candidate identification function 215, based on the role information obtained at step SB2, the progress information obtained at step SB3, the record of messages obtained at step SB4, and/or the medical service information obtained at step SB5 (step SB6). After step SB6, the processing circuitry 21 displays candidate messages identified at step SB6 on a candidate display field, with the display control function 217 (step SB7). According to the second embodiment, candidate messages are identified by further using the medical service information. As a result, candidate messages that more suitably reflect the current conditions can be identified and displayed.

For instance, in the same manner as in the first embodiment, the processing circuitry 21 inputs the role information, progress information, and/or record of messages to the first model to identify candidate messages, extracts a candidate message relating to the medical service information from the identified candidate messages, and displays the extracted candidate message. Clinical cases for different types of medical service information will be described below.

### Case 1: Medical Service Information From Electronic Medical Recording System

The patient basic information is used for various purposes for the care of a stroke-suspected patient. For instance, a patient with a medical history of high blood pressure, diabetes or heart disease has a high risk of developing a stroke. If a patient is taking an anti-clogging medicine, it would be difficult for this patient to undergo hemorrhaging surgery or the like. During a discussion on the input screen regarding, for example the diagnosis of a stroke-suspected patient and the possibility of a surgery on this patient, message candidates may be displayed based on the information obtained from the electronic chart. If the patient's medical history includes a record corresponding to the risk of stroke risk in the past, the processing circuitry 21 may display a candidate message such as “high risk of stroke with high blood pressure and diabetes”.

### Case 2: Medical Service Information From Examination Reservation System

The necessity of a CT examination may be being discussed on the input screen. Only if the medical information processing apparatus 2 receives information reporting that a CT examination order is not issued from the examination reservation system, may the processing circuitry 21 display an option for issuing a CT examination order on the input screen.

### Case 3: Medical Service Information From PACS

Medical images, such as CT images, taken by an imaging apparatus become viewable from various places around the hospital once they are entered into the PACS. For instance, if the medical information processing apparatus 2 receives information that a head CT image of the target patient is newly received from the PACS, the physician who is conducting an image diagnosis will naturally check on this image. The processing circuitry 21 therefore displays an option for displaying the image on the input screen. Furthermore, there may be a situation in which only images with suspected abnormalities may be looked into. In this case, the processing circuitry 21 receives from the PACS the information that a new image has been received, and only if the analysis result from the image processing system is that an abnormality is suspected, may an option for displaying an image be configured to appear on the input screen.

### Case 4: Medical Service Information From Image Processing System

The presence/absence of cerebral hemorrhage or cerebral infarction become a decisive factor for the next medical action. For instance, when the medical information processing apparatus 2 receives the information of the analysis result from the image processing system, which indicates that no cerebral hemorrhage is observed and that cerebral infarction is suspected, it is common to administer t-PA to dissolve blood clots. In this case, the processing circuitry 21 may display an option of “t-PA arrived” on the input screen.

### Case 5: Medical Service Information From Reporting System

The image diagnosis obtained by the radiologist on a well-equipped medical image display workstation tends to produce more accurate results in comparison to the result of images viewed by a neurological physician or the like on a mobile device. If the medical information processing apparatus 2 receives from the reporting system the information that a diagnostic imaging report of the target patient is entered, the processing circuitry 21 may display an option for displaying the image diagnosing result on the input screen. Furthermore, if the history of chatting includes a record of the neurological physician or the like viewing the image on a mobile device, the processing circuitry 21 may further receive from the reporting system the summary of the diagnostic imaging report, and may display an option for displaying the report on the input screen only when the result of the summary is inconsistent with the result of the diagnostic imaging report.

### Case 6: Medical Service Information From Vitals Monitoring System

The vitals data may serve as a crossroads in the treatment of a cerebral stroke. For instance, if the patient has a high blood pressure, cerebral hemorrhage may be caused by the high blood pressure, and if this is the case, the t-PA administration may only worsen the cerebral hemorrhage. For this reason, if the medical information processing apparatus 2 receives the latest blood pressure information from the vitals monitoring system, which indicates a high blood pressure, and the medical information processing apparatus 2 also receives from the medication information system the information that the t-PA is not yet administered, the processing circuitry 21 may perform processing such that an option for administration will not be displayed on the input screen even though the t-PA is not yet administered.

### Case 7: Medical Service Information From Electrocardiograph System

Heart diseases such as atrial fibrillation and cardiac infarction may lead to a cerebral stroke, and therefore electrocardiography may be performed on a stroke-suspected patient. For instance, if information indicating the completion of electrocardiography on the target patient and the presence/absence of an abnormality in the result of the electrocardiography are received from the electrocardiograph system by the medical information processing apparatus 2, the processing circuitry 21 may display a contact button for a cardiologist on the input screen in case the result of the electrocardiography shows an abnormality.

### Case 8: Medical Service Information From Medication Information System

The t-PA is one of the typical medicines for patients suspected of having cerebral infarction to dissolve a clot which causes the cerebral infarction. The medicinal guidelines specify that this medicine should be administered within three hours from the occurrence of the cerebral infarction. Furthermore, the medicine is contraindicated for those who have a history of intracranial hemorrhage, a major surgery within 14 days, or a serious injury at a site other than the head. For instance, even if the medical information processing apparatus 2 receives from the medication information system the information that the t-PA is unadministered, the processing circuitry 21 may perform processing such that an option for t-PA administration will not be displayed on the input screen based on the patient's medical history information from the electronic chart or messages exchanged on the chat indicating that the patient has a contraindication.

After step SB7, the processing circuitry 21 determines, with the display control function 217, whether or not a candidate message displayed at step SB7 is selected (step SB8). If it is determined that a candidate message is selected (yes at step SB8), the processing circuitry 21 displays a message corresponding to the selected candidate message in a message display field of the input screen (step SB9). If it is determined that a candidate message is not selected (no at step SB8), the processing circuitry 21 displays a manually input message in the message display field (step SB10).

After step SB9 or SB10, the processing circuitry 21 terminates the processing of FIG. 12. Thereafter, the processing circuitry 21 waits for an input of a trigger, and upon receipt of a trigger, executes the operations at steps SB1 through SB9 or SB10.

In the above embodiment, the medical service information acquiring process is executed every time a trigger for the presentation of candidate messages is received. There is a concern, however, that an increase in the types of medical service information and the number of information providing systems may lead to a delay in the medical service information acquiring process and analyzing process, eventually hampering immediacy in the presentation of candidate messages and medical service information. In order to ensure the immediacy, the processing circuitry 21 obtains medical service information through either one of push notification, automatic polling, or stepwise processing. In the case of push notification, upon generation of medical service information in the hospital information system 1, the processing circuitry 21 receives the medical service information reported by the hospital information system 1 through push notification, and caches the received medical service information in the storage device 22. In the case of automatic polling, upon receipt of a trigger relating to the presentation of candidate messages, the processing circuitry 21 polls the hospital information system 1 in relation to the obtainment of the medical service information. In the case of stepwise processing, the processing circuitry 21 displays on the input screen the medical service information obtainment status of the systems in the hospital information system 1, namely systems from which the medical service information have been obtained, systems from which the medical service information is now being obtained, and/or systems from which the medical service information has not yet been obtained. The above measures for ensuring the immediacy are not limited to the obtainment of the medical service information, but are also applicable to the obtainment of the role information and/or progress information.

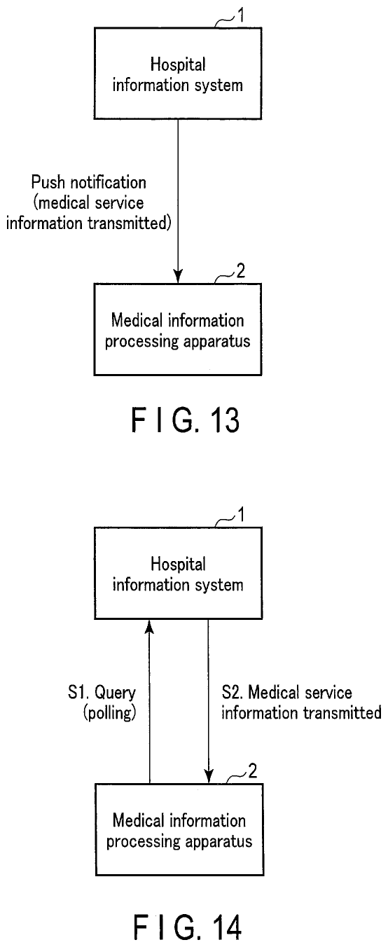

FIG. 13 shows a medical service information acquiring process through push notification. As illustrated in FIG. 13, the medical service information is communicated between the hospital information system 1 and medical information processing apparatus 2. It should be noted that the hospital information system 1 includes various computer systems. For the sake of simplicity, however, a single computer system from which the medical service information is obtained is illustrated as a single block. If a new piece of medical service information regarding the target care is generated at the hospital information system 1, the hospital information system 1 voluntarily transmits this medical service information to the medical information processing apparatus 2 through push notification. The medical information processing apparatus 2 receives the medical service information through the push notification, and caches it in the storage device 22. The cached medical service information is used for identification of subsequent candidate messages.

With the push notification, only when new medical service information is generated at the hospital information system 1, is the medical service information transmitted to the medical information processing apparatus 2. Thus, in comparison with the case of performing the medical service information acquiring process every time a trigger is issued, the amount of data communicated with the systems included in the hospital information system 1 can be reduced. The push notification can therefore serve as a means for ensuring the immediacy of the processing.

FIG. 14 shows a medical service information acquiring process through automatic polling. In the same manner as in FIG. 13, the hospital information system 1 from which the medical service information is obtained is illustrated as a single block in FIG. 14. Upon issuance of a trigger, the medical information processing apparatus 2 first inquires of (polls) the hospital information system 1 as to whether there is any update or addition to the medical service information previously provided to the device 2 (S1). The hospital information system 1 checks the query, and if there is no update or addition, the system 1 responds to the medical information processing apparatus 2 accordingly. In this case, the medical information processing apparatus 2 uses the previously obtained medical service information as-is for the subsequent candidate message identifying process and the like. On the other hand, if there is an update or addition, the hospital information system 1 transmits the medical service information to the medical information processing apparatus 2 (S2). The medical information processing apparatus 2 receives the medical service information and uses the received medical service information for the subsequent candidate message identifying process and the like.

With the automatic polling, an additional process for checking whether or not an update to or addition to the medical service information has been made at the hospital information system 1 needs to be incorporated, in comparison with the push notification. With this technique, however, the communication of the medical service information occurs only in the event of an update or addition, which can reduce the amount of data communicated with various systems of the hospital information system 1. Thus, the automatic polling helps ensure the immediacy of the processing.

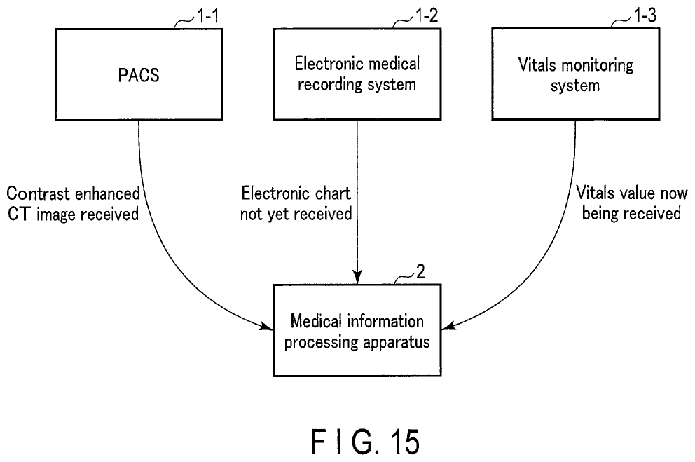

FIG. 15 shows a medical service information acquiring process in accordance with stepwise processing. The hospital information system 1 in FIG. 15 includes the PACS 1-1, electronic medical recording system 1-2, and vitals monitoring system 1-3. At the time of the medical information processing apparatus 2 acquiring the medical service information from the PACS 1-1, electronic medical recording system 1-2, and vitals monitoring system 1-3, there may be slow-response devices (continuously delayed due to hardware performance or the like) or slow-response circumstances (temporarily delayed due to a heavy load being applied to a device). In order to cope with such circumstances, the medical information processing apparatus 2 performs the medical service information acquiring process (SB5), candidate message identifying process (SB6), and candidate message displaying process (SB7) in parallel, on the systems 1-1, 1-2, and 1-3 of the hospital information system 1, respectively.

In the example illustrated in FIG. 15, the medical information processing apparatus 2 has received a contrast enhanced CT image from the PACS 1-1, has not yet received an electronic chart from the electronic medical recording system 1-2, and is now receiving a vitals value from the vitals monitoring system 1-3. In this case, the processing circuitry 21 obtains a “contrast enhanced CT image”, which is the medical service information, and “contrast enhanced CT image received”, which is the progress information; identifies candidate messages based on the obtained information; and displays the identified candidate messages. Here, the processing circuitry 21 displays the progress of the obtainment of the medical service information from the different systems of the hospital information system 1, on the input screen of the chat application.

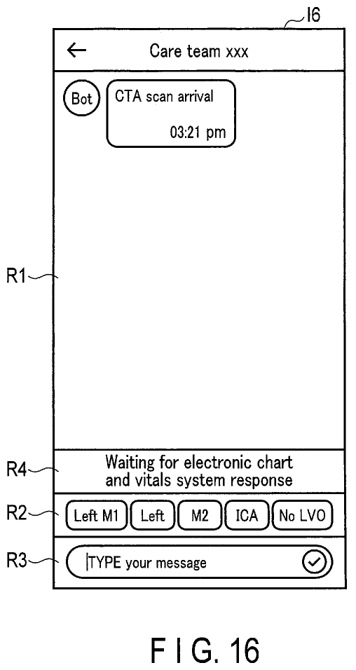

FIG. 16 shows an exemplary display of the progress of medical service information obtainment. The input screen I6 of FIG. 16 corresponds to the input screen I1 of FIG. 6. As illustrated in FIG. 16, the input screen I6 includes a progress status display field R4, in addition to the message display field R1, candidate display field R2, and manual input field R3. The contents displayed in the message display field R1, candidate display field R2, and manual input field R3 are the same as in FIG. 6. The contents displayed in the message display field R1 and candidate display field R2 are based on the contrast enhanced CT image received from the PACS 1-1.

In the progress status display field R4, the progress status regarding the obtainment of the medical service information from different systems of the hospital information system 1 is displayed. For instance, if the medical information processing apparatus 2 is waiting for the medical service information from the electronic medical recording system 1-2 and vitals monitoring system 1-3, a message such as “waiting for electronic chart and vitals system” may be displayed. The display content in the progress status display field R4 is not limited thereto. For instance, information relating to a hospital information system 1 from which the medical service information has been received, such as “information obtained from PACS”, may be displayed. With regard to the medical service information that is being waited for, detailed information such as the medical service information now being received or medical service information yet to be received may be displayed.

In order to enhance the suitability of a candidate message, the processing circuitry 21 may use patient personal information as the medical service information. The patient personal information includes the patient's basic information, medical history, medication history, and/or examination results. The patient's basic information may include the height, weight, age, and allergic state. Application scenarios of the patient personal information will be described below. The application scenarios may be divided into diagnostic confirmation, determination of courses of treatment, and prognosis prediction.

### Application Scenario: Diagnostic Confirmation

For instance, in order to identify the cause of cerebral stroke, the patient's medical history and medication history may need to be referred to. One of the causes of a cerebral stroke is embolization or rupture in a vessel. For patients who have had risk factors such as diabetes or high blood pressure in the past, embolization to a vessel often becomes the cause. For patients who are taking medicine that tends to lead to an abnormality in blood coagulation, embolization often becomes the cause. It is therefore essential to check the patient's medication history.

### Application Scenario: Determination of Courses of Treatment

For cases of complications, for example with high blood pressure and diabetes, the administration dose and type of medicine need to be moderated. The courses of treatment therefore need to be determined in consideration of the patient's medical history, medication history, allergic state and the like. For instance, if a hemorrhage occurs in a patient taking an anticoagulant, the medication needs to be suspended. Thus, the treatment needs to be adjusted in consideration of the patient's medical history, medication history, examination results, and the like.

### Application Scenario: Prognosis Prediction

For patients with an advanced age or with complications, the possibility of a poor prognosis becomes high. The prognosis therefore needs to be predicted in consideration of the patient's age, medical history, medication history, examination results, and the like.

According to the above embodiments, the medical information processing apparatus 2 includes a processing circuitry 21. In the target channel opened on a chat application for discussing a target care, the processing circuitry 21 obtains a record of messages which have been entered by the members of the target channel. The processing circuitry 21 obtains the role information indicating the role of the target member among these members. The processing circuitry 21 obtains the progress information indicating the current progress of the target care. Based on the record of messages, role information, and/or progress information, the processing circuitry 21 identifies one or more candidate messages for a message to be entered by the target member. The processing circuitry 21 displays one or more candidate messages on the input screen of the target channel of the target member.

With the above configuration that displays candidate messages on the input screen, cumbersome tasks for the target member to think of the text of a message or to input such a message can be reduced. This also expedites the decision making among the care team members using the chat application. As a result, the team medical care can be effectively conducted with the chat application even in urgent cases such as a cerebral stroke and pulmonary embolism.

According to at least one of the embodiments described above, swift decision making in team medical care can be reliably assisted.

The term “processor” used in the above description may denote a circuit such as a CPU, GPU, or application specific integrated circuit (ASIC), programmable logic device (e.g., simple programmable logic device (SPLD), complex programmable logic device (CPLD), and field programmable gate array (FPGA)). The processor realizes functions by reading and executing a program stored in the memory circuitry. Instead of storing the program in the memory circuit, the program may be directly incorporated into the circuit of the processor. In this case, the processor realizes the functions by reading and executing the program incorporated into the circuit. If the process is an ASIC, instead of storing the program in the memory circuitry, the functions are directly installed in the circuitry of the processor as a logic circuit. The processors according to the embodiments are not limited to a single circuit for each processor, but may be configured as a single processor by combining different independent circuits to realize the functions. Furthermore, multiple structural elements in FIGS. 1, 2, and 11 may be integrated into one processor to realize their functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising a processing circuitry configured to:
obtain, on a channel opened in a chat application and used for discussing a target care, a record of messages that have been entered by a plurality of members of the channel;
obtain role information indicating a role of a target member among the members;
obtain progress information indicating a current progress of the target care;
identify one or more candidates for a message to be entered by the target member based on the record of messages, the role information, and/or the progress information; and
display the one or more candidates on an input screen of the target member for the channel.

2. The medical information processing apparatus according to claim 1, wherein
each of the one or more candidates is a first candidate for a message that has a function as a character string representing a conversational sentence, a second candidate for a message that has a function to issue a medical procedures order, or a third candidate for a message that has a function to send a query for information.

3. The medical information processing apparatus according to claim 2, wherein
the processing circuitry displays the one or more candidates with a visual effect corresponding to the function of the candidates.

4. The medical information processing apparatus according to claim 1, wherein
if one candidate is selected from the one or more candidates, the processing circuitry displays a message corresponding to the one candidate in a message display field of the input screen.

5. The medical information processing apparatus according to claim 4, wherein
each of the one or more candidates is a first candidate for a message that has a function as a character string representing a conversational sentence, a second candidate for a message that has a function to issue a medical procedures order, or a third candidate for a message that has a function to send a query for information, and
the processing circuitry displays a message corresponding to the one candidate with a visual effect corresponding to the function of the one candidate.

6. The medical information processing apparatus according to claim 1, wherein
if the role information of the target member indicates a radiologist and the progress information indicates completion of image analysis, the processing circuitry displays as the one or more candidates a button for inputting a character string indicating an image diagnosing result.

7. The medical information processing apparatus according to claim 1, wherein
if the role information of the target member indicates a physician specialized in a symptom suspected in the target care, the processing circuitry displays as the one or more candidates a button for ordering a medical procedure to deal with the symptom.

8. The medical information processing apparatus according to claim 7, wherein
if the button is pressed, the processing circuitry places an order corresponding to the button to an order recipient system.

9. The medical information processing apparatus according to claim 1, wherein
if the role information of the target member indicates a physician specialized in a symptom suspected in the target care and the record of messages does not include a message relating to an onset time of the symptom, the processing circuitry displays as the one or more candidates a button for sending a query on the onset time.

10. The medical information processing apparatus according to claim 1, wherein
if the target member is a member newly invited onto the channel, the processing circuitry displays as the one or more candidates a button for sending a query for treatment content information relating to details of the target care.

11. The medical information processing apparatus according to claim 1, wherein
if an alert condition is satisfied on the channel while a task assigned to the target member is incomplete, the processing circuitry displays as the one or more candidates a button for asking details of the task.

12. The medical information processing apparatus according to claim 7, wherein
if the button is pressed, the processing circuitry displays a message corresponding to the button on the input screen.

13. The medical information processing apparatus according to claim 1, wherein
the processing circuitry is further configured to:
obtain medical service information relating to the target care from a hospital information system; and
identify the one or more candidates based further on the medical service information.

14. The medical information processing apparatus according to claim 13, wherein
when a trigger for presenting the one or more candidates occurs, the processing circuitry obtains the medical service information from the hospital information system.

15. The medical information processing apparatus according to claim 13, wherein
the processing circuitry receives the medical service information through a push notification which is performed by the hospital information system upon generation of the medical service information at the hospital information system, and caches the received medical service information in a storage device.

16. The medical information processing apparatus according to claim 13, wherein
when a trigger for presenting the one or more candidates occurs, the processing circuitry polls the hospital information system in relation to obtainment of the medical service information.

17. The medical information processing apparatus according to claim 13, wherein
the processing circuitry displays, on the input screen, a medical service information obtainment status for a system of the hospital information system from which the medical service information has been obtained, a system of the hospital information system from which the medical service information is currently being obtained, and/or a system of the hospital information system from which the medical service information has not yet been obtained.

18. The medical information processing apparatus according to claim 13, wherein
the medical service information includes basic information, medical history, medication history, and/or examination results of a patient relating to the target care.

19. The medical information processing apparatus according to claim 13, wherein
the hospital information system includes an electronic medical recording system, an examination reservation system, a PACS, an image processing system, a reporting system, a vitals monitoring system, an electrocardiographic system, and/or a medication information system.

20. A medical information processing method comprising:
acquiring, on a channel opened in a chat application and used for discussing a target care, a record of messages that have been entered by a plurality of members of the channel;
acquiring role information indicating a role of a target member among the members;
acquiring progress information indicating a current progress of the target care;
identifying one or more candidates for a message to be entered by the target member based on the record of messages, the role information, and/or the progress information; and
displaying the one or more candidates on an input screen of the target member for the channel.
